(19) United States
(12) Patent Application Publication
(10) Pub. No.: US 2014/0031679 A1
(43) Pub. Date: Jan. 30, 2014

(54) OPTICAL SCANNING PROBE

(71) Applicants: Yoshiyuki Tashiro, Tokyo (JP); Masashi Kitatsuji, Tokyo (JP); Seiichi Yokoyama, Tokyo (JP)

(72) Inventors: Yoshiyuki Tashiro, Tokyo (JP); Masashi Kitatsuji, Tokyo (JP); Seiichi Yokoyama, Tokyo (JP)

(73) Assignee: HOYA CORPORATION, Tokyo (JP)

(21) Appl. No.: 14/111,019

(22) PCT Filed: Jan. 23, 2012
(86) PCT No.: PCT/JP2012/051314

§ 371 Date: Oct. 10, 2013

(30) Foreign Application Priority Data
Apr. 26, 2011 (JP) 2011-098016

(51) Int. Cl. A61B 5/00

(52) U.S. Cl. CPC A61B 5/0077
USPC 600/425

(57) ABSTRACT

An optical scanning probe comprising a flexible tube, an optical fiber for transmitting scanning light that is supported in the flexible tube to be able to freely rotate about an axis of the optical fiber, and an objective lens that has a positive optical power to convert the scanning light emerging from the optical fiber from a divergent beam to a collimated beam or a convergent beam and rotates integrally with the optical fiber, and wherein the objective lens is provided with a deflection surface that deflects the scanning light to irradiate an object.

### TECHNICAL FIELD

The present invention relates an optical scanning probe for optically scanning an object.

### BACKGROUND ART

As an imaging system for imaging body tissue inside a lumen, an optical scanning system is known. As an example of a specific configuration of an optical scanning system, OCT (Optical Coherence Tomography) for observing in detail a fine structure near a surface layer of a lumen such as a digestive organ or a bronchial tube is described, for example, in Japanese Patent Provisional Publication No. HEI 11-56786A (hereafter, referred to as patent document 1).

The OCT system described in the patent document 1 includes an OCT probe to be inserted into a lumen. The OCT probe described in the patent document 1 irradiates a side wall of the lumen with low coherence light by transmitting the low coherence light emitted from a light source through an optical fiber. In accordance with rotation of the optical fiber about an axis thereof, the low coherence light scans on the side wall of the lumen in a circumferential direction. The OCT system measures the position and depth at which scanning light is reflected or scattered in the lumen and the degree of reflection or scattering of the scanning light in the lumen based on the principle of low coherence interferometry, and calculates and generates tomographic image data of the lumen using measurement results. The generated tomographic image of the lumen has a higher resolution than that of a tomographic image generated by an ultrasonic system and the like that is typically used at present.

### SUMMARY OF THE INVENTION

In the OCT probe described in the patent document 1, a GRIN lens that focuses low coherence light is coupled to a tip of the optical fiber. A microprism that bends an optical path of the low coherence light toward the side wall of the lumen is bonded to a tip surface of the GRIN lens. Since this kind of microprism is a minute optical part, it has a problem that it is difficult to process. Furthermore, since scattered light from a subject such as the side wall of the lumen is in general very weak, there is a demand that a loss of light amount that depends on optical systems is suppressed as much as possible.

The present invention is made in view of the above described circumstances. The object of the invention is to provide an optical scanning probe suitable to simplify the manufacture and to suppress the loss of light amount that depends on optical systems.

To solve the above described problem, an optical scanning probe according to an embodiment of the invention comprises: a flexible tube; an optical fiber for transmitting scanning light that is supported in the flexible tube to be able to freely rotate about an axis of the optical fiber; and an objective lens that has a positive optical power to convert the scanning light emerging from the optical fiber from a divergent beam to a collimated beam or a convergent beam and rotates integrally with the optical fiber. The objective lens according to the invention has a deflection surface that deflects the scanning light to irradiate an object.

According to the invention, a microprism, a component that is minute and difficult to process, conventionally considered as an essential component in an optical scanning probe, is not necessary. Therefore, manufacturing becomes easier and the loss of light amount of scanning light is suppressed thanks to reduction of a transmission surface for the scanning light (reduction of a joint surface of between a microprism and a GRIN lens conventionally used), as well as reduction of the number of components and man-hour.

The objective lens is, for example, a GRIN lens. The deflection surface of the GRIN lens may be an object side end surface of the GRIN lens formed to be inclined with respect to the axis.

The deflection surface of the objective lens may be a cylindrical surface that has predetermined curvature in one direction. The predetermined curvature of the cylindrical surface may be set to a value that corrects astigmatism caused when the scanning light transmits through the GRIN lens and the flexible tube.

The deflection surface of the objective lens may be a reflective surface provided with a coating which reflects the scanning light or a total reflection surface that totally reflects the scanning light.

The optical scanning probe according to the invention may comprise a barycenter adjustment member that is bonded to the deflection surface of the objective lens and causes a combined barycenter of the objective lens and the barycenter adjustment member to be situated on the axis of the optical fiber.

According to the invention, an optical scanning probe suitable for suppressing loss of light amount that depends on optical systems, as well as for simplifying the manufacturing is provided.

### BRIEF DESCRIPTION OF THE DRAWINGS

FIG. 1 is a block diagram illustrating a configuration of an OCT system according to an embodiment of the invention.

FIG. 2 illustrates an internal configuration of an OCT probe according to the embodiment of the invention.

FIG. 3 illustrates an outer shape of a GRIN lens provided in the OCT probe according to the embodiment of the invention.

FIG. 4 illustrates an outer shape of a GRIN lens provided in an OCT probe according to another embodiment of the invention.

FIG. 5 illustrates an internal configuration of an OCT probe according to another embodiment of the invention.

### EMBODIMENTS FOR CARRYING OUT THE INVENTION

In the following, an optical scanning system having an optical scanning probe according to the invention is explained with reference to the accompanying drawings. In this embodiment, as a specific configuration of an optical scanning system, an OCT system that performs measurement based on the principle of low coherence interferometry and generates an image using the measurement data is given as an example.

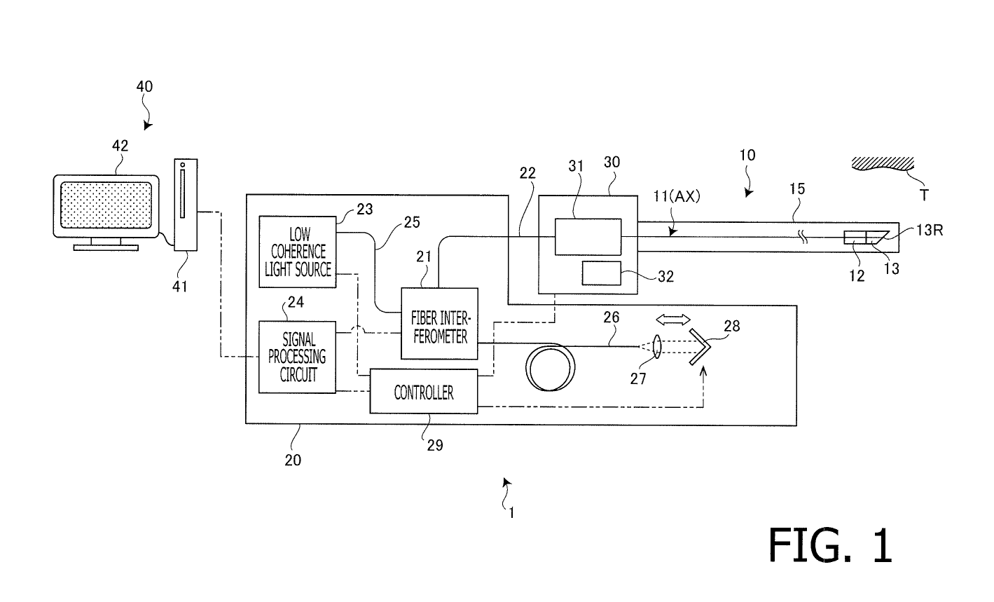

FIG. 1 is a block diagram illustrating a general configuration of OCT system 1. In FIG. 1, a path of an electric signal is represented by a double chain line, an optical path of an optical fiber is represented by a solid line and an optical path of light proceeding through air or a living tissue is represented by a dashed line respectively. In the following explanation, in regard to an optical path in the OCT system 1, a side closer to a light source is defined as a proximal side, and a side farther from the light source is defined as a tip side.

As shown in FIG. 1, the OCT system 1 has an OCT probe 10 for obtaining an image near a surface layer of a lumen T, such as a digestive organ or a bronchial tube. The OCT probe 10 is connected to a system main unit 20 via a probe scanning device 30. Specifically, the probe scanning device 30 optically connects a proximal end of an optical fiber 11 of the OCT probe 10 with a tip of a probe optical fiber 22 extending to the outside of the system main unit 20 from a fiber interferometer 21 of the system main unit 20. In FIG. 1, for simplification of the diagram, a configuration of the OCT probe 10 is represented by minimum elements required for explaining the principle of OCT observation. Furthermore, for convenience of explanation, the center axis (which coincides with the rotation center axis of the optical fiber 11 in design) of the OCT probe 10 is referred to as a “reference axis AX”.

In addition to the fiber interferometer 21 and the probe optical fiber 22, the system main unit 20 has a low coherence light source 23, a signal processing circuit 24, a supply optical fiber 25, a reference optical fiber 26, a lens 27, a dach mirror 28 and a controller 29. The controller 29 totally executes various types of control of the OCT system 1, such as light emission control of the low coherence light source 23, control of the signal processing circuit 24 and driving of motors for the dach mirror 28 and the probe scanning device 30.

The low coherence light source 23 is a light source being able to emit low coherence light, and specifically the low coherence light source 23 is a SLD (Super Luminescent Diode). The low coherence light emitted from the low coherence light source 23 is incident on the proximal end of the supply optical fiber 25. The supply optical fiber 25 transmits the low coherence light being incident thereon to the fiber interferometer 21. The fiber interferometer 21 divides the low coherence light from the supply optical fiber 25 into two optical paths with a component such as an optical coupler. One of the divided optical paths propagates through the probe optical fiber 22 as object light. The other of the divided optical paths propagates through the reference optical fiber 26 as reference light.

The probe scanning device 30 has a rotary joint 31 which couples the tip of the probe optical fiber 22 with the proximal end of the optical fiber 11. To the rotary joint 31, a radial scan motor 32 is connected via a transmission mechanism not shown. In accordance with driving of the radial scan motor 32, the rotary joint 31 rotates the optical fiber 11 about the reference axis AX, with respect to the probe optical fiber 22. The object light transmitted through the probe optical fiber 22 is incident on the proximal end of the optical fiber 11 via the rotary joint 31.

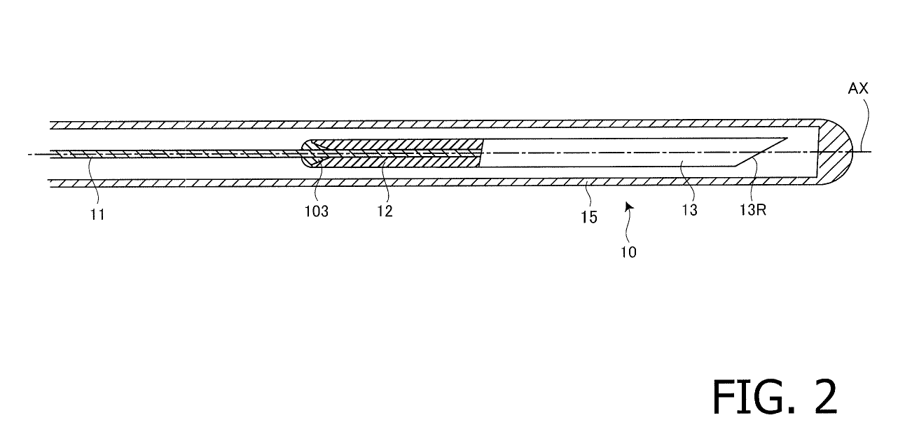

FIG. 2 illustrates an inner configuration of the OCT probe 10. As shown in FIG. 2, the OCT probe 10 has the optical fiber 11, a ferrule 12 and a GRIN lens 13. Each of components including the optical fiber 11, the ferrule 12 and the GRIN lens 13 has a substantially cylindrical shape, and is accommodated in a tube-shaped outer sheath 15 forming an outer appearance of the OCT probe 10. The outer sheath 15 is formed of flexible materials so that the OCT probe 10 can be inserted into a lumen.

The optical fiber 11 is supported inside the ferrule 12 along the reference axis AX, and is bonded using a thermoset adhesive 103. A tip surface of the optical fiber 11 is set at the same plane as a tip surface of the ferrule 12, and is connected optically and mechanically to the GRIN lens 13.

The object light incident on the proximal end of the optical fiber 11 propagates through the optical fiber 11 and is incident on the GRIN lens 13. A deflection surface 13R of the GRIN lens 13 is inclined with respect to the reference axis AX, and is coated with a metal film such as aluminum to reflect the object light.

The object light is incident on and reflected by the area around the point where the reference axis AX intersects with the deflection surface 13R, while being converged from divergent beam to collimated or convergent beam by the GRIN lens 13 having positive optical power. The object light of which optical path is bent by the reflection propagates through the outer sheath 15 and is emitted toward a side wall of the lumen T. At least an optical path between the GRIN lens 13 and the outer sheath 15 is filled with a fluid such as silicone oil to suppress loss of light amount due to the difference in refractive index.

The outer circumferential surface of the GRIN lens 13, from which the object light bent by the deflection surface 13R is emitted, acts as a cylindrical surface since the GRIN lens 13 has a cylindrical shape. Furthermore, since the outer sheath 15 is tube-shaped, the inner and outer circumferential surfaces of the outer sheath 15 through which the object light transmits act as cylindrical surfaces as well. Therefore, astigmatism arises.

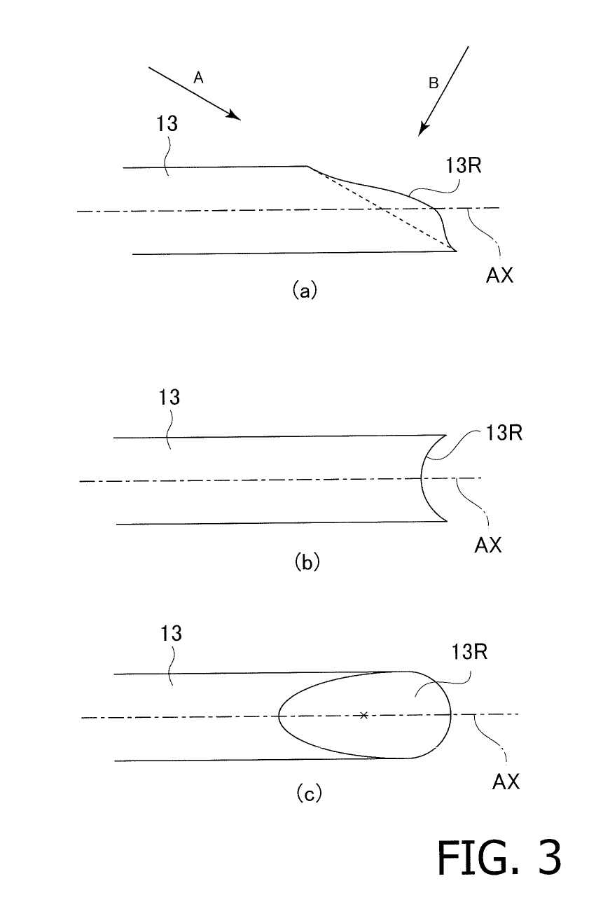

For this reason, the deflection surface 13R has a predetermined cylindrical shape to cancel out the astigmatism caused by the object light transmission surfaces of the GRIN lens 13 and the outer sheath 15. FIG. 3 (a) illustrates a side view of an outer appearance of the GRIN lens 13. FIGS. 3 (b) and (c) illustrate outer appearances of the GRIN lens 13 viewed along the directions indicated by arrows A and B in FIG. 3 (a) respectively. As shown in FIG. 3, the deflection surface 13R has curvature, by which the deflection surface 13R is formed in appearance to be a concave shape, in a direction (referred to as a “sagittal plane direction” for convenience) perpendicular to the reference axis AX, and has no curvature in a direction (referred to as a “meridional plane direction” for convenience) perpendicular to the sagittal plane direction. Therefore, a relative position of a sagittal image plane with respect to a meridional image plane of the object light can be controlled by the curvature of the cylindrical surface of the deflection surface 13R, and thus astigmatism can be reduced. By thus designing the cylindrical surface of the deflection surface 13R in the aforementioned way, both the meridional and sagittal image planes can be adjusted to the vicinity of the image plane position of the GRIN lens 13 itself (in this case, a meridional image plane position), facilitating calculations necessary for design and thereby giving advantages in the designing phase.

The GRIN lens 13 is fixed to the optical fiber 11 along with the ferrule 12. Consequently, the whole configuration from the optical fiber 11 to the GRIN lens 13 rotate integrally about the reference axis AX as the radial scan motor 32 is activated. Herewith the object light scans the lumen T in the circumferential direction.

As the low coherence light, near infrared light having a property of propagating through a living tissue relative to visible light is generally used. The object light reaches a portion near the surface layer of the lumen T, and is reflected or scattered. Then, a part of the reflected or scattered object light is incident on the GRIN lens 13. A returned light which is incident on the GRIN lens 13 returns to the fiber interferometer 21 via the optical fiber 11, the rotary joint 31 and the probe optical fiber 22.

The reference light propagates through the reference optical fiber 26, emits from the tip of the reference optical fiber 26 and is incident on a lens 27. The lens 27 converts the reference light from a divergent beam to a collimated beam, and lets the collimated beam to emerge therefrom. The dach mirror 28 reflects the collimated beam emerging from the lens 27 to be incident on the lens 27 again. In order to make an optical path length of the reference light changeable, the dach mirror 28 is supported to be able to freely move in the optical axis direction (a direction of an arrow in FIG. 1) by a driving mechanism not shown. The reference light sent back to the lens 27 returns to the fiber interferometer 21 via the reference optical fiber 26.

In the fiber interferometer 21, measurement of an interferometric signal using the principle of a low coherence interferometer is performed. Specifically, in the fiber interferometer 21, an interferometric signal is obtained only when optical path lengths of the object light returned from the probe optical fiber 22 and the reference light returned from the reference optical fiber 26 are approximately equal to each other. The intensity of the interferometric signal is determined depending on a degree of reflection or scattering of the object light occurred at a particular position of the lumen T (the optical path length of the object light) corresponding to the position of the dach mirror 28 (the optical path length of the reference light).

The fiber interferometer 21 outputs, to the signal processing circuit 24, the interferometric signal corresponding to an interference pattern of the object light and the reference light. The signal processing circuit 24 executes a predetermined process for the inputted interferometric signal, and assigns a pixel address to the interferometric signal depending on a scanning position of the interferometric signal. The scanning position in the circumferential direction of the lumen T is identified by a driving amount of the radial scan motor 32, and the scanning position in the depth direction of the lumen T is identified by the driving amount of a drive motor (not shown) of the dach mirror 28.

The signal processing circuit 24 performs buffering, into a frame memory not shown by frame basis, for a signal of an image constituted by a spatial arrangement of point images represented by the interferometric signals in accordance with the assigned pixel addresses. The buffered signal is swept out from the frame memory at predetermined timing, and is outputted to an information processing terminal 41 of a display device 40. The information processing terminal 41 executes a predetermined process for the inputted signal and converts the inputted signal into a video signal, and displays an image near the surface layer of the lumen T on a monitor 42.

In the OCT probe 10 according to the embodiment, by omitting a minute microprism from the system, manufacturing is facilitated since reflection surface processing can be applied to the GRIN lens 13 which is bigger than a microprism, in addition to reduction of the number of components and man-hour. Furthermore, the loss of light amount of object light is suppressed thanks to reduction of a transmission surface for the object light (reduction of a joint surface of between a microprism and a GRIN lens conventionally used).

The foregoing is the explanation of the embodiment of the invention. The invention is not limited to the above described configuration, and can be varied within the scope of the technical concept of the invention. For example, in addition to the OCT system of TD-OCT (Time Domain OCT) type, the invention can be applied to an OCT system of FD-OCT (Fourier Domain OCT) type, such as SD-OCT (Spectral Domain OCT) type or SS-OCT (Swept Source OCT) type.

In a case where a refractive index of a medium outside the deflection surface 13R is less than the GRIN lens 13, like air, the deflection surface 13R may be a total reflection surface without particular processes for reflection.

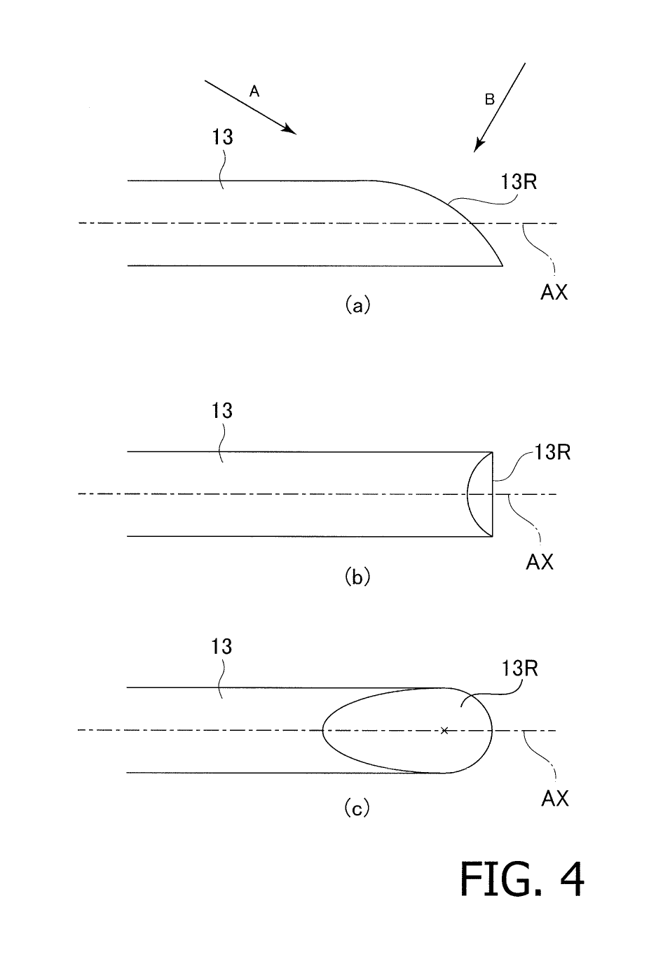

FIG. 4 (a) illustrates a side view of an outer appearance of the GRIN lens 13 according to another embodiment of the invention. FIGS. 4 (b) and (c) illustrate the appearance of the GRIN lens 13 taken along the directions indicated by arrows A and B in FIG. 4 (a), respectively. As shown in FIG. 4, the deflection surface 13R in this embodiment has curvature in the meridional plane direction making it a convex shape in appearance, and has no curvature in the sagittal plane direction. Therefore, the relative position of the meridional image plane with respect to the sagittal image plane of the object light can be controlled by curvature of a cylindrical surface, and thus astigmatism can be reduced. By designing a cylindrical surface of the deflection surface 13R in the aforementioned way, the deflection surface 13R can cover a part of optical power that the GRIN lens 13 should provide totally, and consequently the overall length of the GRIN lens 13 can be designed shorter. Is becomes easier to insert the OCT probe in a lumen since the length of non-flexible portion of the OCT probe 10 becomes shorter.

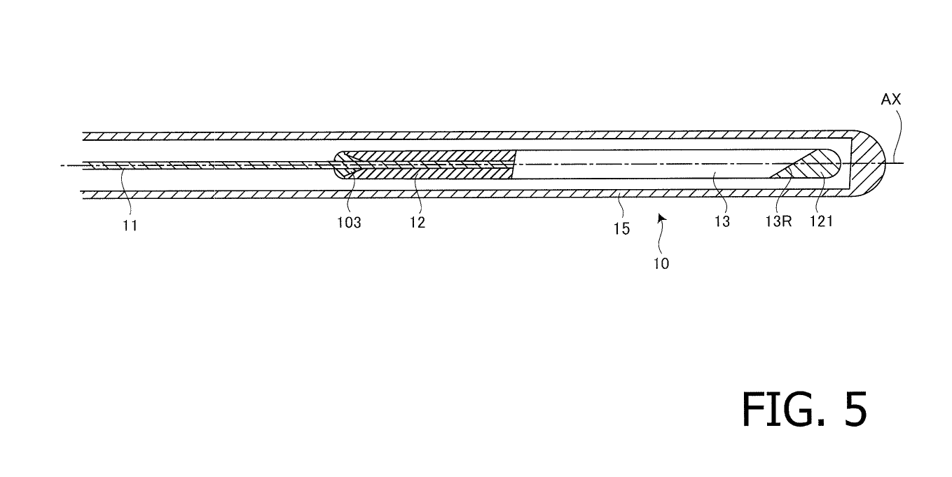

FIG. 5 illustrates an internal configuration of an OCT probe 10 according to yet another embodiment of the invention. In FIG. 5, to elements which are the same as or similar to those of the OCT probe 10 in FIG. 2, the same reference numbers are assigned, and explanations thereof will be simplified or omitted.

The barycenter of the GRIN lens 13 is shifted from the reference axis AX. Therefore, the tip of the optical fiber 11 and the GRIN lens 13 produce a swinging motion about the reference axis AX when the driving force of the radial scan motor 32 is transmitted thereto. Hence, as shown in FIG. 5, a barycenter adjustment member 121 is bonded to the backside of the deflection surface 13R in the OCT probe 10 in this embodiment. The OCT probe 10 shown in FIG. 5 has the same configuration as that of the OCT probe 10 shown in FIG. 2, except that the barycenter adjustment member 121 is bonded to the backside of the deflection surface 13R.

The GRIN lens 13 and the barycenter adjustment member 121 are made of the same material or of materials having substantially the same specific gravity. Therefore, the combined barycenter of the GRIN lens 13 and the barycenter adjustment member 121 is on the reference axis AX. Since the combined barycenter of all the parts bonded to the tip of the optical fiber 11 (the ferrule 12, the GRIN lens 13, and the barycenter adjustment member 121) is on the rotation center axis of the optical fiber 11, the tip portion of the optical fiber 11 stably rotates approximately on the reference axis AX. As a result, the position of the deflection surface 13R with respect to the reference axis AX becomes stable, and thereby the focal point also becomes stable.

The volume, material and specific gravity of the barycenter adjustment member 121 are not limited as long as the combined barycenter of the GRIN lens 13 and the barycenter adjustment member 121 is located on the reference axis AX and the rotation movement thereof inside the outer sheath 15 is not hampered.

There is a concern about an erosion phenomenon by cavitation when a component is rotated at a high speed in a fluid having a high degree of viscosity, such as silicon oil. For this reason, the shape of the barycenter adjustment member 121 is based on a cylindrical shape having substantially the same diameter as that of the GRIN lens 13, and the proximal end surface of the barycenter adjustment member 121 has a shape corresponding to the deflection surface 13R (transferred shape of the deflection surface 13R). Since the GRIN lens 13 and the barycenter adjustment member 121 is bonded to be coaxial, edges of both members (the edge of the deflection surface 13R and the edge of the proximal end surface of the barycenter adjustment member 121) do not appear on the outer shape contour. Furthermore, the tip edge of the barycenter adjustment member 121 is chamfered in a shape of a curved surface. That is, since no edge appears on the outer shape contour, there is no part having a high fluid resistance during rotational motion, and thereby occurrence of the cavitation can be effectively suppressed.

Since the barycenter adjustment member 121 is bonded to the GRIN lens 13, the barycenter adjustment member 121 also has a function of protecting the deflection surface 13R.

1. An optical scanning probe, comprising:
a flexible tube;
an optical fiber for transmitting scanning light that is supported in the flexible tube to be able to freely rotate about an axis of the optical fiber; and
an objective lens that has a positive optical power to convert the scanning light emerging from the optical fiber from a divergent beam to one of a collimated beam and a convergent beam and rotates integrally with the optical fiber,
wherein the objective lens has a deflection surface that deflects the scanning light to irradiate an object.

2. The optical scanning probe according to claim 1, wherein the objective lens is a GRIN lens.

3. The optical scanning probe according to claim 2, wherein the deflection surface is an object side end surface of the GRIN lens formed to be inclined with respect to the axis.

4. The optical scanning probe according to claim 1, wherein the deflection surface is a cylindrical surface that has predetermined curvature in one direction.

5. The optical scanning probe according to claim 4, wherein the predetermined curvature of the cylindrical surface is set to a value that corrects astigmatism caused when the scanning light transmits through the objective lens and the flexible tube.

6. The optical scanning probe according to claim 1, wherein the deflection surface is one of a reflective surface provided with a coating which reflects the scanning light and a total reflection surface that totally reflects the scanning light.

7. The optical scanning probe according to claim 1, further comprising a barycenter adjustment member that is bonded to the deflection surface and causes a combined barycenter of the objective lens and the barycenter adjustment member to be situated on the axis of the optical fiber.
